(19) United States
(12) Patent Application Publication
(10) Pub. No.: US 2026/0137869 A1
(43) Pub. Date: May 21, 2026

(54) CELL DELIVERY INJECTOR

(71) Applicant: BAYER HEALTHCARE LLC, Whippany, NJ (US)

(72) Inventors: Ankur-Aaron SHARMA, Sewickley, PA (US); Arthur E. UBER, III, Pittsburgh, PA (US); David GRIFFITHS, Pittsburgh, PA (US)

(21) Appl. No.: 19/119,582

(22) PCT Filed: Nov. 13, 2023
(86) PCT No.: PCT/US2023/037202

§ 371 Date: Apr. 9, 2025

Related U.S. Application Data
(60) Provisional application No. 63/425,031, filed on Nov. 14, 2022.

(51) Int. Cl. A61M 5/172; A61M 5/145; A61M 5/168; G16H 20/17

(52) U.S. Cl. CPC A61M 5/1723; A61M 5/145; A61M 5/16804; A61M 5/16831; A61M 5/16877; G16H 20/17; A61M 2202/0411; A61M 2202/0429; A61M 2205/3303; A61M 2205/3331; A61M 2205/3368

(57) ABSTRACT

Provided herein is a system for delivering a therapeutic composition to a patient, including: a container configured to hold a therapeutic composition; at least one powered drive in operative connection with the container; a plurality of sensors including at least two of (i) a pressure sensor, (ii) a flow sensor, (iii) a counting sensor, (iv) a site sensor, (v) a temperature sensor, (vi) a chemical sensor, and (vii) at least one physiological sensor; and at least one processor programmed or configured to receive the data from the plurality of sensors and, control the at least one powered drive to inject the therapeutic composition into the patient at a desired flow rate whose magnitude depends on the data received from the plurality of sensors.

### CROSS-REFERENCE TO RELATED APPLICATIONS

The present application claims the benefit of priority to U.S. Provisional Ser. No. 63/425,031 , filed Nov. 14, 2022, the content of which is incorporated herein by reference in its entirety.

### BACKGROUND OF THE INVENTION

### Field of the Invention

The present disclosure relates to devices, systems, and methods for delivery of cell-based therapies.

### Description of Related Art

The treatment of disease by the injection of living cells into a body is expanding rapidly. There are many types of cells being used to treat an equally diverse set of diseases, and both types of cells and disease conditions are expanding rapidly. Cell therapy is the transfer of intact, live cells into a patient to help lessen or cure a disease. The cells may originate from the patient (autologous cells) or a donor (allogeneic cells), some of which may be genetically modified. Some examples of cell therapy include CAR-T cell therapy, in which T cells genetically engineered to express chimeric antigen receptors (CARs) are delivered to patients, for example in patients with certain subtypes of B cell leukemia or lymphoma. Promising efficacy of cell therapy has also been demonstrated in patients with multiple myeloma. However, various barriers restrict the efficacy and/or prevent the widespread use of cell therapies. These barriers include cell rupture, which can cause dangerous side effects in patients. Accordingly, there is a need in the art for more robust delivery devices and systems that can account and correct for various conditions that may arise during any such cell delivery treatments.

### SUMMARY OF THE INVENTION

Provided herein is a system for delivering a cell therapy to a patient, including: a container configured to hold a therapeutic composition, the therapeutic composition including one or more cells in a suspension; at least one powered drive in operative connection with the container, the at least one powered drive being configured to pressurize the therapeutic composition within the container and at least one fluid line in communication therewith; a control system having a plurality of sensors and at least one processor programmed or configured to receive data from the plurality of sensors and, based at least in part on the data, to control the at least one powered drive so as to pressurize the therapeutic composition within the container and the at least one fluid line and thereby to inject the therapeutic composition into the patient at a first flow rate; the plurality of sensors including at least two of a pressure sensor for measuring and providing data indicative of pressure within the container and the at least one fluid line, a flow sensor for measuring and providing data indicative of an actual flow rate of the suspension within the at least one fluid line, a counting sensor for providing data indicative of a count within the suspension flowing through the at least one fluid line of at least one of (i) a number of the one or more cells that remain whole and (ii) a number of the one or more cells that have ruptured, a site sensor for placement about a site of injection of the suspension into the patient and for providing data indicative of one of an occurrence and a non-occurrence of an extravasation thereat, a temperature sensor for measuring and providing data indicative of a temperature of the suspension within the at least one fluid line, a chemical sensor for detecting and providing data indicative of an occurrence of an immune response in the patient, and at least one physiological sensor for measuring and providing data indicative of at least one parameter of the patient, wherein the at least one processor is further programmed or configured to control the at least one powered drive to pressurize the therapeutic composition within the container and thereby to inject the therapeutic composition into the patient at a second flow rate upon determining that the data received from the plurality of sensors indicate at least one of: the pressure within at least one of the container and the at least one fluid line is greater than a predetermined pressure threshold, the flow rate of the suspension within the at least one fluid line is greater than a predetermined flow rate threshold, the number of the one or more cells within the suspension that have ruptured is greater than a predetermined rupture threshold, the occurrence of an extravasation threat about the site of the injection, the temperature of the suspension within the at least one fluid line is outside a predetermined temperature range, the occurrence of an immune response in the patient as evidenced by the data received from at least one of the chemical sensor and the at least one physiological sensor, and an adverse reaction experienced by the patient as evidenced by the data received from the at least one physiological sensor.

Also provided herein is a system for delivering a cell therapy to a patient, including: a container configured to hold a therapeutic composition, the therapeutic composition including one or more cells in a suspension; at least one powered drive in operative connection with the container, the at least one powered drive being configured to pressurize the therapeutic composition within the container and at least one fluid line in communication therewith; a plurality of sensors including at least two of (i) a pressure sensor for measuring and providing data indicative of pressure within the container and the at least one fluid line, (ii) a flow sensor for measuring and providing data indicative of an actual flow rate of the suspension within the at least one fluid line, (iii) a counting sensor for providing data indicative of a count within the suspension flowing through the at least one fluid line of at least one of (i) a number of the one or more cells that remain whole and (ii) a number of the one or more cells that have ruptured, (iv) a site sensor for placement about a site of injection of the suspension into the patient and for providing data indicative of one of an occurrence and a non-occurrence of an extravasation thereat, (v) a temperature sensor for measuring and providing data indicative of a temperature of the suspension within the at least one fluid line, (vi) a chemical sensor for detecting and providing data indicative of an occurrence of an immune response in the patient, and (vii) at least one physiological sensor for measuring and providing data indicative of at least one parameter of the patient; and at least one processor programmed or configured to receive the data from the plurality of sensors and, based at least in part on the data, to control the at least one powered drive so as to pressurize the therapeutic composition within the container and the at least one fluid line and thereby to inject the therapeutic composition into the patient at a desired flow rate whose magnitude depends on the data received from the plurality of sensors.

Also provided herein is a system for delivering a cell therapy to a patient, including: a container configured to hold a therapeutic composition including one or more autologous and/or allogenic cells and an ultrasound contrast agent; a pump configured to expel the therapeutic composition from the container; at least one processor in communication with the pump; and a plurality of sensors in communication with the at least one processor, the plurality of sensors including at least: a counting sensor configured to collect cell data wherein the cell data includes a number of whole and/or ruptured cells, a pressure sensor configured to collect pressure data wherein the pressure data includes a measurement of pressure within the container and/or one or more fluid lines between the container and the patient, a flow sensor configured to collect flow data wherein the flow data includes a measurement of flow rate within one or more fluid lines between the container and the patient, an ultrasound transducer configured to be placed about a site of injection of the therapeutic composition into the patient and to collect extravasation data wherein the extravasation data is indicative of one of an occurrence and a non-occurrence of an extravasation thereat, at least one physiological sensor configured to collect physiological data wherein the physiological data includes measurement of an immune parameter, blood pressure, heart rate, respiratory rate, and/or temperature; wherein the at least one processor is programmed or configured to control the pump to pressurize the therapeutic composition to deliver the therapeutic composition at a first flow rate, and determine, based at least in part on the cell data, the pressure data, the flow data, the extravasation data, and the physiological data, that the therapeutic composition should be delivered at the first flow rate or the therapeutic composition should be delivered at a second flow rate.

Additional non-limiting aspects are described below:

In a first aspect, a system for delivering a cell therapy to a patient is provided, including a container configured to hold a therapeutic composition, the therapeutic composition including one or more cells in a suspension; at least one powered drive in operative connection with the container, the at least one powered drive being configured to pressurize the therapeutic composition within the container and at least one fluid line in communication therewith; a control system having a plurality of sensors and at least one processor programmed or configured to receive data from the plurality of sensors and, based at least in part on the data, to control the at least one powered drive so as to pressurize the therapeutic composition within the container and the at least one fluid line and thereby to inject the therapeutic composition into the patient at a first flow rate; the plurality of sensors including at least two of: a pressure sensor for measuring and providing data indicative of pressure within the container and the at least one fluid line; a flow sensor for measuring and providing data indicative of an actual flow rate of the suspension within the at least one fluid line; a counting sensor for providing data indicative of a count within the suspension flowing through the at least one fluid line of at least one of (i) a number of the one or more cells that remain whole and (ii) a number of the one or more cells that have ruptured; a site sensor for placement about a site of injection of the suspension into the patient and for providing data indicative of one of an occurrence and a non-occurrence of an extravasation thereat; a temperature sensor for measuring and providing data indicative of a temperature of the suspension within the at least one fluid line; a chemical sensor for detecting and providing data indicative of an occurrence of an immune response in the patient; and at least one physiological sensor for measuring and providing data indicative of at least one parameter of the patient; wherein the at least one processor is further programmed or configured to control the at least one powered drive to pressurize the therapeutic composition within the container and thereby to inject the therapeutic composition into the patient at a second flow rate upon determining that the data received from the plurality of sensors indicate at least one of: the pressure within at least one of the container and the at least one fluid line is greater than a predetermined pressure threshold; the flow rate of the suspension within the at least one fluid line is greater than a predetermined flow rate threshold; the number of the one or more cells within the suspension that have ruptured is greater than a predetermined rupture threshold; the occurrence of an extravasation threat about the site of the injection; the temperature of the suspension within the at least one fluid line is outside a predetermined temperature range; the occurrence of an immune response in the patient as evidenced by the data received from at least one of the chemical sensor and the at least one physiological sensor; and an adverse reaction experienced by the patient as evidenced by the data received from the at least one physiological sensor.

In a second aspect, which may be based on the first aspect, the container is one of a syringe, a bag, a bottle and a vial.

In a third aspect, which may be based on the first aspect and/or the second aspect, the one or more cells include at least one of: (i) autologous cells, (ii) allogenic cells, (iii) a combination of the autologous cells and the allogenic cells, (iv) a mixture in which at least one of the autologous cells and the allogenic cells have been modified, (v) genetically-engineered cells and (vi) genetically-engineered T-cells that express chimeric antigen receptors.

In a fourth aspect, which may be based on any of the foregoing aspects, one of the plurality of sensors is an ultrasound sensor and the suspension includes an ultrasound contrast agent.

In a fifth aspect, which may be based on any of the foregoing aspects, the at least one powered drive is a pump system including an injector system.

In a sixth aspect, which may be based on any of the foregoing aspects, the second flow rate is one of a cessation of flow and a reduction in flow.

In a seventh aspect, which may be based on any of the foregoing aspects, the at least one physiological sensor includes at least one of a blood pressure sensor, a heart rate sensor, a respiration sensor, a temperature sensor, and a chemical sensor.

In an eighth aspect, which may be based on any of the foregoing aspects, upon determining that the patient is experiencing an adverse reaction, the at least one processor is programmed or configured to cause the at least one powered drive to deliver a second therapeutic composition to the patient.

In a ninth aspect, which may be based on any of the foregoing aspects, the second therapeutic composition includes at least one of a crystalloid solution, a colloid solution, and a corticosteroid, wherein the crystalloid solution includes at least one of normal saline, D5W, and lactated Ringers.

In a tenth aspect, which may be based on any of the foregoing aspects, the at least one processor is further programmed or configured to trigger at least one of an alarm and an alert upon determining that the data received from the plurality of sensors indicate any of: the pressure within at least one of the container and the at least one fluid line is greater than the predetermined pressure threshold; the flow rate of the suspension within the at least one fluid line is greater than the predetermined flow rate threshold; the number of the one or more cells within the suspension that have ruptured is greater than the predetermined rupture threshold; the occurrence of an extravasation about the site of the injection; the temperature of the suspension within the at least one fluid line is outside a predetermined temperature range; the occurrence of an immune response in the patient as evidenced by the data received from at least one of the chemical sensor and the at least one physiological sensor; and an adverse reaction experienced by the patient as evidenced by the data received from the at least one physiological sensor.

In an eleventh aspect, which may be based on any of the foregoing aspects, the system includes at least one flow regulator positioned within the at least one fluid line, each of the at least one flow regulators being configured to be switchable between an open state and a closed state such that upon: determining that a volume of the therapeutic composition needs to be increased, the at least one processor is programmed or configured to switch the at least one flow regulator to the open state and thereby permit at least one of a diluent and a buffer to be added to the suspension via the at least one fluid line; determining that the volume of the therapeutic composition needs to be reduced, the at least one processor is programmed or configured to switch the at least one flow regulator to the open state and thereby permit a portion of fluid within the suspension to be removed therefrom via the at least one fluid line; and detecting that the number of the one or more cells that have ruptured has reached the predetermined rupture threshold, the at least one processor is programmed or configured to switch the at least one flow regulator to the open state and thereby divert the suspension to a separate fluid line of the at least one fluid line for at least one of analysis and disposal.

In a twelfth aspect, provided is a system for delivering a cell therapy to a patient, including a container configured to hold a therapeutic composition, the therapeutic composition including one or more cells in a suspension; at least one powered drive in operative connection with the container, the at least one powered drive being configured to pressurize the therapeutic composition within the container and at least one fluid line in communication therewith; a plurality of sensors including at least two of (i) a pressure sensor for measuring and providing data indicative of pressure within the container and the at least one fluid line; (ii) a flow sensor for measuring and providing data indicative of an actual flow rate of the suspension within the at least one fluid line; (iii) a counting sensor for providing data indicative of a count within the suspension flowing through the at least one fluid line of at least one of (i) a number of the one or more cells that remain whole and (ii) a number of the one or more cells that have ruptured; (iv) a site sensor for placement about a site of injection of the suspension into the patient and for providing data indicative of one of an occurrence and a non-occurrence of an extravasation thereat; (v) a temperature sensor for measuring and providing data indicative of a temperature of the suspension within the at least one fluid line; (vi) a chemical sensor for detecting and providing data indicative of an occurrence of an immune response in the patient; and (vii) at least one physiological sensor for measuring and providing data indicative of at least one parameter of the patient; and at least one processor programmed or configured to receive the data from the plurality of sensors and, based at least in part on the data, to control the at least one powered drive so as to pressurize the therapeutic composition within the container and the at least one fluid line and thereby to inject the therapeutic composition into the patient at a desired flow rate whose magnitude depends on the data received from the plurality of sensors.

In a thirteenth aspect, which may be based on any of the foregoing aspects, the desired flow rate is initially a first flow rate but changes to a second flow rate when the data received by the at least one processor from the plurality of sensors indicate at least one of: the pressure within at least one of the container and the at least one fluid line is greater than a predetermined pressure threshold; the flow rate of the suspension within the at least one fluid line is greater than a predetermined flow rate threshold; the number of the one or more cells within the suspension that have ruptured is greater than a predetermined rupture threshold; the occurrence of an extravasation about the site of the injection; the temperature of the suspension within the at least one fluid line is outside a predetermined temperature range; the occurrence of an immune response in the patient as evidenced by the data received from at least one of the chemical sensor and the at least one physiological sensor; and an adverse reaction experienced by the patient as evidenced by the data received from the at least one physiological sensor.

In a fourteenth aspect, which may be based on any of the foregoing aspects, the one or more cells include at least one of (i) autologous cells, (ii) allogenic cells, (iii) a combination of the autologous cells and the allogenic cells, (iv) a mixture in which at least one of the autologous cells and the allogenic cells have been modified, (v) genetically-engineered cells and (vi) genetically-engineered T-cells that express chimeric antigen receptors.

In a fifteenth aspect, which may be based on any of the foregoing aspects, one of the plurality of sensors is an ultrasound sensor and the suspension includes an ultrasound contrast agent.

In a sixteenth aspect, which may be based on any of the foregoing aspects, the at least one powered drive is a pump system including an injector system.

In a seventeenth aspect, which may be based on any of the foregoing aspects, the second flow rate is one of a cessation of flow and a reduction in flow.

In an eighteenth aspect, which may be based on any of the foregoing aspects, the at least one physiological sensor includes at least one of a blood pressure sensor, a heart rate sensor, a respiration sensor, a temperature sensor and a chemical sensor.

In a nineteenth aspect, which may be based on any of the foregoing aspects, upon determining that the patient is experiencing an adverse reaction, the at least one processor is programmed or configured to cause the at least one powered drive to deliver a second therapeutic composition to the patient.

In a twentieth aspect, which may be based on any of the foregoing aspects, the second therapeutic composition includes at least one of a crystalloid solution, a colloid solution, and a corticosteroid, wherein the crystalloid solution includes at least one of normal saline, D5W, and lactated Ringers.

In a twenty-first aspect, which may be based on any of the foregoing aspects, the at least one processor is further programmed or configured to trigger at least one of an alarm and an alert upon determining that the data received from the plurality of sensors indicate any of: the pressure within at least one of the container and the at least one fluid line is greater than a predetermined pressure threshold; the flow rate of the suspension within the at least one fluid line is greater than a predetermined flow rate threshold; the number of the one or more cells within the suspension that have ruptured is greater than a predetermined rupture threshold; the occurrence of an extravasation about the site of the injection; the temperature of the suspension within the at least one fluid line is outside a predetermined temperature range; the occurrence of an immune response in the patient as evidenced by the data received from at least one of the chemical sensor and the at least one physiological sensor; and an adverse reaction experienced by the patient as evidenced by the data received from the at least one physiological sensor.

In a twenty-second aspect, which may be based on any of the foregoing aspects, the system further includes at least one flow regulator positioned within the at least one fluid line, each of the at least one flow regulator being configured to be switchable between an open state and a closed state such that upon: determining that a volume of the therapeutic composition needs to be increased, the at least one processor is programmed or configured to switch the at least one flow regulator to the open state and thereby permit at least one of a diluent and a buffer to be added to the suspension via the at least one fluid line; determining that the volume of the therapeutic composition needs to be reduced, the at least one processor is programmed or configured to switch the at least one flow regulator to the open state and thereby permit a portion of fluid within the suspension to be removed therefrom via the at least one fluid line; and detecting that the number of the one or more cells that have ruptured has reached a predetermined rupture threshold, the at least one processor is programmed or configured to switch the at least one flow regulator to the open state and thereby divert the suspension to a separate fluid line of the at least one fluid line for at least one of analysis and disposal.

In a twenty-third aspect, provided is a system for delivering a cell therapy to a patient, including: a container configured to hold a therapeutic composition including one or more autologous and/or allogenic cells and an ultrasound contrast agent; a pump configured to expel the therapeutic composition from the container; at least one processor in communication with the pump; and a plurality of sensors in communication with the at least one processor, the plurality of sensors including at least: a counting sensor configured to collect cell data, the cell data including a number of whole and/or ruptured cells; a pressure sensor configured to collect pressure data, the pressure data including a measurement of pressure within the container and/or one or more fluid lines between the container and the patient; a flow sensor configured to collect flow data, the flow data including a measurement of flow rate within one or more fluid lines between the container and the patient; an ultrasound transducer configured to be placed about a site of injection of the therapeutic composition into the patient and to collect extravasation data, the extravasation data indicative of one of an occurrence and a non-occurrence of an extravasation thereat; at least one physiological sensor configured to collect physiological data, the physiological data including measurement of an immune parameter, blood pressure, heart rate, respiratory rate, and/or temperature, wherein the at least one processor is programmed or configured to: control the pump to pressurize the therapeutic composition to deliver the therapeutic composition at a first flow rate; and determine, based at least in part on the cell data, the pressure data, the flow data, the extravasation data, and the physiological data, that: the therapeutic composition should be delivered at the first flow rate; or the therapeutic composition should be delivered at a second flow rate.

In a twenty-fourth aspect, which may be based on any of the foregoing aspects, the determination is based at least on: a determination that the pressure and/or the flow rate is too great based at least in part on the pressure data and/or the flow data; a determination that an extravasation threat is occurring based at least in part on the extravasation data, a determination that greater than a predetermined threshold of cells has ruptured based at least in part on the cell data; and/or a determination that the patient is experiencing an adverse reaction based at least in part on the physiological data.

In a twenty-fifth aspect, which may be based on any of the foregoing aspects, upon determining that: at least one of the flow rate and the pressure is too great; an extravasation threat is occurring; a threshold number of cells has ruptured; and/or the patient is experiencing an adverse reaction, the at least one processor is further programmed or configured to control the pump to deliver the therapeutic composition at the second flow rate.

In a twenty-sixth aspect, which may be based on any of the foregoing aspects, the second flow rate is one of a cessation of flow and a reduction in flow.

In a twenty-seventh aspect, which may be based on any of the foregoing aspects, upon determining that the flow and/or the pressure is too great, that there is extravasation, that a threshold number of cells has ruptured, and/or that the patient is experiencing an adverse reaction, the at least one processor is programmed or configured to trigger an alert or an alarm.

In a twenty-eighth aspect, which may be based on any of the foregoing aspects, the alarm is an audible alarm, a visual alarm, and/or a tactile alarm.

In a twenty-ninth aspect, which may be based on any of the foregoing aspects, upon determining that the patient is experiencing an adverse reaction, the at least one processor is further programmed or configured to cause the pump to deliver a second therapeutic composition to the patient.

In a thirtieth aspect, which may be based on any of the foregoing aspects, the second therapeutic composition includes at least one of a crystalloid solution, a colloid solution, and a corticosteroid, wherein the crystalloid solution includes at least one of normal saline, D5W, and lactated Ringers.

In a thirty-first aspect, which may be based on any of the foregoing aspects, the container is one of a syringe, a bag, a bottle and a vial.

### BRIEF DESCRIPTION OF THE DRAWINGS

FIG. 1 is a diagram of a non-limiting embodiment of an environment in which devices, systems, and/or methods described herein may be implemented;

FIG. 2 is a diagram of a non-limiting aspect or embodiment of components of one or more devices and/or one or more systems of FIG. 1; and

FIG. 3 is a flowchart of a non-limiting embodiment of a process for delivering a therapeutic to a patient.

### DESCRIPTION OF THE INVENTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosure as it is oriented in the drawing figures. However, it is to be understood that the disclosure may assume various alternative variations and step sequences, except where expressly specified to the contrary. It is also to be understood that the specific devices and processes illustrated in the attached drawings, and described in the following specification, are simply exemplary embodiments or aspects of the disclosure. Hence, specific dimensions and other physical characteristics related to the embodiments or aspects of the embodiments disclosed herein are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or send (e.g., transmit) information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively send information to the second unit. As another example, a first unit may be in communication with a second unit if at least one intermediary unit (e.g., a third unit located between the first unit and the second unit) processes information received from the first unit and sends the processed information to the second unit. In some non-limiting embodiments, a message may refer to a network packet (e.g., a data packet and/or the like) that includes data.

As used herein, the term “computing device” may refer to one or more electronic devices that are configured to directly or indirectly communicate with or over one or more networks. In some non-limiting embodiments, a computing device may include a mobile device. A mobile device may include a smartphone, a portable computer, a wearable device (e.g., watches, glasses, lenses, clothing, and/or the like), a personal digital assistant (PDA), and/or other like devices. In some non-limiting embodiments, a computing device may include a server, a desktop computer, and/or the like.

As used herein, the term “system” may refer to one or more computing devices or combinations of computing devices such as, but not limited to, processors, servers, client devices, software applications, and/or other like components. In addition, reference to “a server” or “a processor,” as used herein, may refer to a previously-recited server and/or processor that is recited as performing a previous step or function, a different server and/or processor, and/or a combination of servers and/or processors. For example, as used in the specification and the claims, a first server and/or a first processor that is recited as performing a first step or function may refer to the same or different server and/or a processor recited as performing a second step or function.

Provided herein are devices, systems, and methods for delivering a therapeutic, such as one or more cells in a suspension, to a patient. Devices, systems, and methods as described herein provide a technological solution to problems in the field of therapeutic delivery, by integrating a plurality of sensors and/or data, through an algorithm executed by a processor, that accounts for a multitude of issues that may be experienced during therapeutic delivery, which cannot currently be adequately accounted for in a simultaneous manner. The devices, systems, and methods described herein improve the functioning of existing delivery devices, such as cell delivery devices, and improve clinical outcomes.

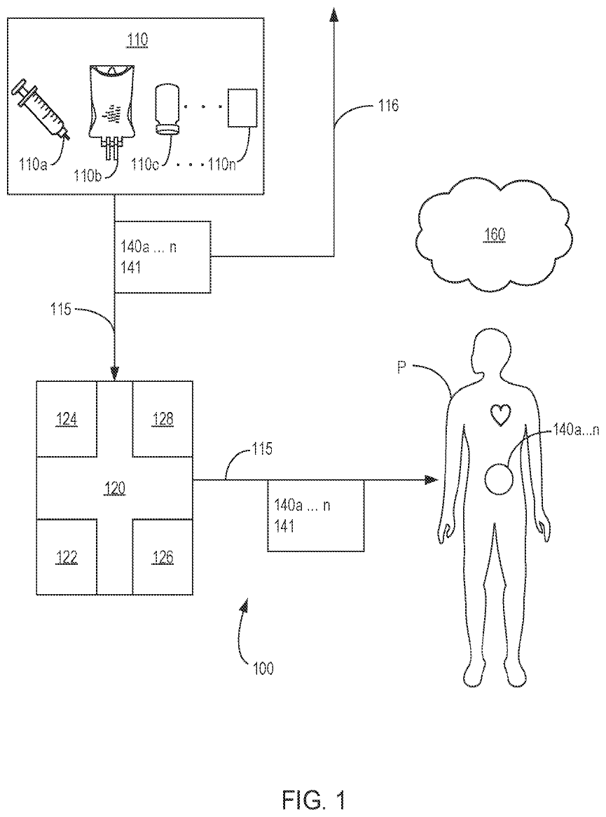

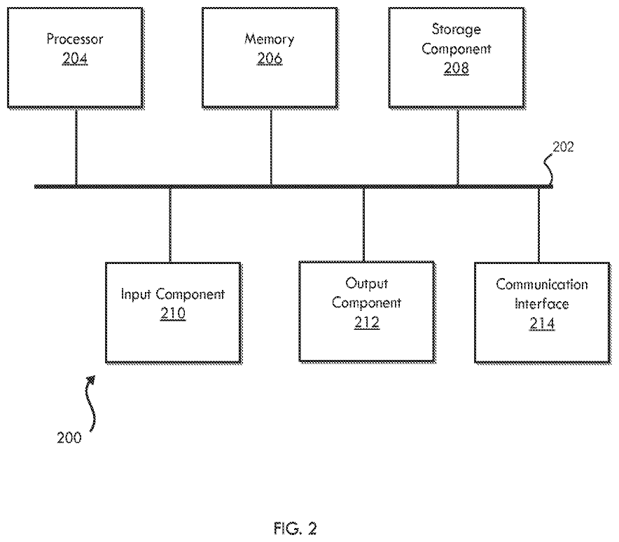

Turning to FIG. 1, in non-limiting embodiments, provided herein is a system 100 including a container 110 configured to hold a therapeutic composition, a delivery device 120, and one or more sensors 140. Delivery device 120 and container 110, while illustrated as separate components connected by fluid line(s) 115, may be integrated. System 100 further includes a powered drive 122 configured to deliver the therapeutic composition to a patient P. Again, while illustrated as a part of delivery device 120, powered drive 122 may be arranged within container 110. In non-limiting embodiments, powered drive 122 includes a pump system or other type of fluid pressurization system. Such a pump system may include an injection system, a gravity-fed system, and/or any combination of components. Delivery device 120 may further include a processor 124, memory 126 to store programming instructions to be executed by processor 124, and a communications interface 128. Aspects of processor 124, memory 126, and communications interface 128 are described below in reference to FIG. 2. System 100 further includes one or more sensors 140. While a plurality of sensors 140 are shown in FIG. 1 in particular arrangements and orientations, those of skill will understand that the number and arrangement of sensors 140 may be modified to achieve the goals of the devices, systems, and methods disclosed herein. Sensors 140 may be in communication with processor 124, such that data detected by the sensors 140 may be received and analyzed by processor 124, for example based on programming instructions communicated to and/or stored in memory 126.

Container 110 may be a syringe 110a, intravenous bag 100b, vial 110c, bottle, or any other vessel 110n capable of holding a therapeutic composition for delivery to a patient P. Container 110 may be lined with one or more suitable coatings to reduce adhesion of the therapeutic composition to one or more surfaces within container 110. In non-limiting embodiments, container 110 may include or be associated with one or more sensors 140, for example to monitor a status of a therapeutic composition received within container 110, during loading, shipping, and use, for example use through system 100. In non-limiting embodiments, such sensors 140 may include optical sensors, force sensors, and/or temperature sensors. In non-limiting embodiments, such optical sensors are configured to permit analysis of one or more cells received within container 110, for example by detection of one or more trackers (e.g., radiolabeling, iron oxide nanoparticles, gallium), cell morphology, and/or digital pathology. Non-invasive tracking methods are known to those of skill in the art, for example as disclosed in Kircher et al., “Noninvasive cell-tracking methods,” Nature Reviews Clinical Oncology 2011, 8:677-688.

Processors associated with system 100 as described herein may make use of data received from sensor(s) 140 to control delivery of the therapeutic composition received within container 110, as described herein. In non-limiting embodiments, a processor associated with system 100, receiving data from one or more sensors 140, may determine whether delivery of the therapeutic composition in container 110 should begin, based on data collected by sensor(s) 140 during loading and/or shipping. For example, if a therapeutic composition received within container 110 was exposed to forces, chemical conditions (pH), temperatures, mean kinetic temperatures, and/or the like that were outside of a predetermined range of acceptable parameters (such as, for example, about 40 degrees Celsius to about 50 degrees Celsius, above about 50 degrees Celsius, and/or between 43 degrees Celsius and 50 degrees Celsius, all values and subranges therebetween inclusive), for example above a certain threshold for a greater than a threshold period of time, and thus that the therapeutic composition should not be delivered to a patient, processor 124 may provide an alert, for example an audible, visual, and/or tactile alert, indicating that delivery of the therapeutic composition should not be initiated. In non-limiting embodiments, upon determining that that the therapeutic composition should not be delivered to a patient, processor 124 may prevent initiation of delivery of the therapeutic composition, and, in non-limiting embodiments, prevention of delivery may or may not be overridden by, for example, entry of a password or other authentication.

In non-limiting embodiments, container 110 may include one or more ports to allow withdrawal of a portion of a therapeutic composition received therein for one or more external analyses. Non-limiting examples of such analyses include microplate assays, such as alamarBlue®, PrestoBlue®, CyQUANT®, and MTT assays, including those available from ThermoFisher Scientific (Watham, MA). Other analyses include fluorescence and/or dye tests (including those described in Kim et al., “Application of a non-hazardous vital dye for cell counting with automated cell counters”, Analy. Biochem. 2016, 492(2): 8-12), visual examination through use of a camera, and/or other analyses suitable for determining a state of a therapeutic composition, for example one or more cells, as known to those of skill in the art.

In non-limiting embodiments, fluid line(s) 115 may be flexible and/or rigid, and may include one or more than one lumen therein. In non-limiting embodiments, container 110 and/or fluid line(s) 115 may include one or more flow regulators 141, such as valves and/or one or more agitators, such as those described in U.S. Pat. No. 6,575,930, the content of which is incorporated herein by reference in its entirety. In non-limiting embodiments, fluid line(s) 115 include one or more needles arranged an end thereof to aid in delivery of the therapeutic composition to the patient P. Suitable needles are known to those of skill in the art, and may include any useful features, depending on the therapeutic composition to be delivered, including various bevels.

In non-limiting embodiments, the therapeutic composition is one or more cells in a suspension. Suitable suspensions for maintaining viability of cells are known to those of skill in the art. In non-limiting embodiments, the suspension includes one or more ultrasound contrast agents, to allow for detection of extravasation, as will be described below. In non-limiting embodiments, extravasation is monitored audibly, by measuring sound from bubbles injected into tissue near the injection site for the cell suspension being destroyed, e.g., by popping. In non-limiting embodiments, extravasation is monitored based upon an absence of an ultrasound return signal from bubble destruction at or proximal to the injection site. In addition to extravasation, other parameters may also be monitored with ultrasound, for example, blood pH as a measure of, for example, inflammation may be measured with ultrasound (see, e.g., Walker et al., “Dynamic solid-state ultrasound contrast agent for monitoring pH fluctuations in vivo”, ACS Sens. 2020, vol. 5, no. 4, pp. 1190-1197). In non-limiting embodiments, the therapeutic composition includes one or more autologous cells, one or more allogenic cells, and/or one or more genetically-engineered cells, such as chimeric antigen receptor T cells (CAR-T cells). In non-limiting embodiments, the therapeutic composition includes autologous cells, allogenic cells, a combination of autologous cells and allogenic cells, a mixture in which at least one of the autologous cells and the allogenic cells have been modified, genetically-engineered cells, and/or CAR-T cells.

With continuing reference to FIG. 1, in non-limiting embodiments sensors 140 include one or more of a counting sensor 140a configured to count whole and/or ruptured cells, a pressure sensor 140b configured to detect pressure within container 110 and/or one or more fluid lines 115, a flow sensor 140c configured to detect flow rate within one or more fluid lines 115, a site sensor 140d configured to detect extravasation, and/or a physiological sensor 140e configured to detect one or more physiological characteristics of the patient P, and/or any other type of sensor 140n. In non-limiting embodiments, cell counters, pressure, and/or flow sensors may be arranged in or on container 110 and/or fluid line(s) 115. As used herein, the term “ruptured cell” refers to cells that are damaged, non-viable, corrupted or otherwise unable to contribute to the desire therapeutic response of the patients.

Suitable sensor(s) for counting whole and/or ruptured cells are known to those of skill in the art, and may include those described in https://www.nist.gov/programs-projects/cell-counting-cell-therapies. In non-limiting embodiments, suitable sensors for counting include those that measure hemolysis as an indirect measure of cell viability, optionally by measuring conductivity of the cell suspension (see, e.g., Van Buren et al., “A simple method to monitor hemolysis in real time,” Sci Rep. 2020; 10, 5101; e.g., Zhuo et al., “Optofluidic sensor for inline hemolysis detection on whole blood”, ACS Sens. 2018, vol. 3, no. 4, pp. 784-791).

Sensor(s) 140 configured to detect flow rate, viscosity, and/or pressure may be useful in the determination of a microenvironment to which the therapeutic composition is exposed, which may be useful for various analyses and processes as described herein, for example to determine forces applied to the therapeutic composition, such as shear forces, which may negatively impact the therapeutic composition (for example, by causing an unacceptably high degree of cell rupture). In non-limiting embodiments, one or more sensors 140 may measure one or more parameters of the therapeutic composition exiting container 110 and/or passing through fluid line(s) 115, and processor 124 may, upon receipt of data from sensor(s) 140 (and, in non-limiting embodiments, based at least in part on diameter of fluid line(s) 115), adjust one or more parameters of delivery of the therapeutic composition, such as, without limitation, flow rate and/or viscosity of the therapeutic composition. In non-limiting embodiments, one or more sensors 140 may measure viscosity of the therapeutic composition exiting container 110 and/or passing through fluid line(s) 115, and processor 124 may, upon receipt of viscosity data from sensor(s) 140, adjust viscosity of the therapeutic composition, for example by adding a less viscous fluid (e.g., a buffer and/or saline) or causing removal of a less viscous fluid (e.g., a buffer and/or saline) from container 110 and/or fluid line(s) 115. In non-limiting embodiments, viscosity may be adjusted through use of siphon lines (e.g., to separate fluid from cell-containing fluid).

In non-limiting embodiments, one or more sensors 140, for example physiological sensors, may be arranged in or on patient P. In non-limiting embodiments, sensor 140 is a site sensor (e.g., a sensor configured to be placed on or near a site of introduction of the therapeutic composition into the patient), and may be a sensor configured to detect extravasation. Sensors and systems for detecting extravasation are known to those of skill in the art, and are described, for example, in Hirata et al., Sensing Technologies for Extravasation Detection: a Review, ACS Sens, 2023, 8:1017-1032. Suitable systems and sensors may include ultrasound, optical sensors, microbubbles, and the like. In non-limiting embodiments, a sensor 140 configured to detect extravasation is an ultrasound transducer, and, optionally, the therapeutic composition being delivered includes an ultrasound contrast medium. In non-limiting embodiments, a sensor 140 configured to detect extravasation is an optical sensor (for example, sensors sold by ivWatch LLC, Newport News, VA). In non-limiting embodiments, one or more of the sensors 140 is a sensor configured to detect surface tension and/or rigidity at the injection site on the patient, which can be used to evaluate extravasation.

In non-limiting embodiments, the one or more physiological sensors is one or more of a sensor for detecting the patient's blood pressure, blood pH, heart rate (including ECG/EKG), oxygen saturation (including pulse oximeters), white blood cell count (including non-invasive white blood cell count sensors, such as those developed by Leuko Labs, Inc., Boston, MA), brain activity (including EEG), pupil dilation, respiratory rate, vocalization (e.g., a microphone), motion, and/or temperature (including perspiration and/or skin flushing). Those of skill in the art will appreciate that delivery of any therapeutic composition, such as a cell therapy, such as CAR-T therapeutic, may be accompanied by an adverse event, which may elicit an immune response, such as an allergic reaction, a cytokine storm (which may also be referred to as cytokine release syndrome), and/or anaphylaxis, and that any suitable sensors for detecting such an adverse event may be used here, including chemical sensors for detecting one or more parameters indicative of an immune response, including, without limitation, sensors for detecting levels of histamine, cytokines, mast cells, immunoglobulins, c-reactive protein (CRP), D-dimer, and growth factors. In non-limiting embodiments, the chemical sensor is an electrochemical sensor. Suitable sensors for detecting an immune response include those described in Xu, et al., Real-Time Monitoring and Early Warning of a Cytokine Storm In Vivo Using a Wearable Noninvasive Skin Microneedle Patch, Advanced Healthcare Materials 2023, 12(18): e2203133.

In non-limiting embodiments, system 100 includes a communication network 160, to provide connectivity between various components of the system, for example between sensor(s) 140, flow regulator(s) 141, and delivery device 120. Communication network 160 may include one or more wired and/or wireless networks. For example, communication network 160 may include a cellular network (e.g., a long-term evolution (LTE) network, a third generation (3G) network, a fourth generation (4G) network, a fifth generation (5G) network, a sixth generation (6G) network, a code division multiple access (CDMA) network, and/or the like), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of some or all of these or other types of networks.

The number and arrangement of systems and/or devices shown in FIG. 1 are provided as an example. There may be additional systems and/or devices, fewer systems and/or devices, different systems and/or devices, or differently arranged systems and/or devices than those shown in FIG. 1. Furthermore, two or more systems and/or devices shown in FIG. 1 may be implemented within a single system or a single device, or a single system or a single device shown in FIG. 1 may be implemented as multiple, distributed systems or devices. Additionally or alternatively, a set of systems or a set of devices (e.g., one or more systems, one or more devices) of system 100 may perform one or more functions described as being performed by another set of systems or another set of devices of system 100.

Referring now to FIG. 2, illustrated is a diagram of example components of device 200. Device 200 may correspond to cell delivery device 120 and/or communication network 160 (e.g., one or more devices of communication network 160). In some non-limiting embodiments or aspects, cell delivery device 120 and/or communication network 160 may include at least one device 200 and/or at least one component of device 200. As illustrated in FIG. 2, device 200 may include bus 202, processor 204, memory 206, storage component 208, input component 210, output component 212, and/or communication interface 214.

Bus 202 may include a component that permits communication among the components of device 200. In some non-limiting embodiments or aspects, processor 204 may be implemented in hardware, software, or a combination of hardware and software. For example, processor 204 may include a processor (e.g., a central processing unit (CPU), a graphics processing unit (GPU), an accelerated processing unit (APU), and/or the like), a microprocessor, a digital signal processor (DSP), and/or any processing component (e.g., a field-programmable gate array (FPGA), an application-specific integrated circuit (ASIC), and/or the like) that can be programmed to perform a function. Memory 206 may include random access memory (RAM), read-only memory (ROM), and/or another type of dynamic or static storage device (e.g., flash memory, magnetic memory, optical memory, and/or the like) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, and/or the like). Additionally or alternatively, input component 210 may include a sensor for sensing information (e.g., a global positioning system (GPS) component, an accelerometer, a gyroscope, an actuator, and/or the like). Output component 212 may include a component that provides output information from device 200 (e.g., a display, a speaker, one or more light-emitting diodes (LEDs), and/or the like).

Communication interface 214 may include a transceiver-like component (e.g., a transceiver, a separate receiver and transmitter, and/or the like) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments or aspects described herein are not limited to any specific combination of hardware circuitry and software.

Memory 206 and/or storage component 208 may include data storage or one or more data structures (e.g., a database, and/or the like). Device 200 may be capable of retrieving information from, storing information in, or searching information stored in the data storage or one or more data structures in memory 206 and/or storage component 208. For example, the information may include encryption data, input data, output data, transaction data, account data, or any combination thereof.

The number and arrangement of components shown in FIG. 2 are provided as an example. In some non-limiting embodiments or aspects, device 200 may include additional components, fewer components, different components, or differently arranged components than those shown in FIG. 2. Additionally or alternatively, a set of components (e.g., one or more components) of device 200 may perform one or more functions described as being performed by another set of components of device 200.

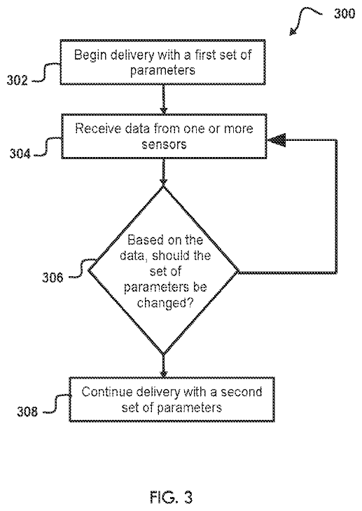

With reference to FIGS. 1 and 3, in non-limiting embodiments, delivery device 120, through processor 124, controls delivery of the therapeutic composition (FIGS. 3, 300). In non-limiting embodiments, delivery device 120 controls powered drive 122 to deliver the therapeutic composition at a first flow rate (FIGS. 3, 302), which may be a desired flow rate. Delivery device 120, through communication between processor 124 and one or more sensors 140 (FIGS. 3, 304) and between processor 124 and powered drive 122, can modulate the flow rate, pressure, viscosity, and/or the like based on data received from the sensor(s) (FIGS. 3, 306). Adjustments in operation of the system 100 may be based on, without limitation, detection that the pressure within the container 110 and/or one or fluid line(s) 115 is greater than a predetermined pressure threshold, determination that the flow rate of the therapeutic composition within the fluid line(s) 115 is greater than a predetermined flow rate threshold, that the number of cells within the therapeutic composition that have ruptured is greater than a predetermined rupture threshold, that an extravasation is occurring or is threatened about the site of the injection, that the temperature of the therapeutic composition within the fluid line(s) 115 is outside a predetermined temperature range, that an immune response is occurring or may occur within the patient, and/or that the patient is or may occur within the patient. For example, when data suggesting that a number of ruptured cells exceeds a predetermined threshold (optionally stored in memory 126), that pressure in container 110 or fluid line(s) 115 is too great, that flow rate in fluid line(s) 115 is too high, that the therapeutic composition has leaked out of the injection site (extravasation), and/or that the patient P is experiencing an adverse event, such as an immune response, the flow rate may be reduced to a second flow rate (FIGS. 3, 308), which may be a desired flow rate, and, in non-limiting embodiments, that second flow rate may be zero (e.g., a cessation of delivery of the therapeutic composition). Those of skill in the art will appreciate that various algorithms may be utilized to determine whether flow rate should be reduced, including, for example and without limitation, application of various weighting factors and/or various thresholds. In non-limiting embodiments, one or more useful algorithms may relate to shear forces and/or any parameter described herein in terms of cell viability, delivery, patient monitoring, and/or patient action.

While cell rupture is exemplified above, predetermined thresholds and/or ranges may be provided for any parameter for which a sensor is provided, with ranges/thresholds stored in memory 126 in non-limiting embodiments. In non-limiting embodiments, when data received from the one or more sensors 140 is within a suitable range, delivery of the therapeutic composition may continue at the first flow rate, with data collection continuing (FIGS. 3, 304). In cases where the therapeutic composition is below the concentration expected or desired, for example if slightly increased but not unsafe levels of ruptured cells, are sensed before or during an injection, the total volume and/or flow rate may be increased so that sufficient total dose is delivered to achieve the desired therapeutic delivery and thus therapeutic response. In this example, the second flow rate many be higher than the first. In non-limiting embodiments, a flow rate may be decreased (e.g., the second flow rate may be lower than the first flow rate), but an increased volume of therapeutic composition may be delivered (for example, as described below), for example by delivering the therapeutic composition for an increased amount of time.

In non-limiting embodiments, in addition to adjusting the flow rate, delivery device 120 may trigger an alarm if any predetermined threshold is exceeded, such that flow is reduced. Suitable alarms may be audible, visual, and/or tactile. Alarms may be presented on delivery device 120 (which may have a user interface, such as a display) and/or, by virtue of delivery device 120 including a communications interface 128, may be transmitted to a device associated with a healthcare professional, such as a nurse, technician, or physician.

In non-limiting embodiments, as an addendum or alternative to adjusting the flow rate, system 100 may be configured, based on processor 124, to adjust the volume of the therapeutic composition. In non-limiting embodiments, the system may be configured to divert a portion of the therapeutic composition out of fluid line(s) 115, for testing and/or disposal, and/or add a buffer and/or diluent to fluid line(s) 115. For example, and without limitation, as shown in FIG. 1, system 100 may be configured such that at one or more positions along fluid line(s) 115, a flow regulator 141 may be arranged, which may be configured to have an open state (e.g., in which fluid may pass through flow regulator 141 and continue along fluid line(s) 115), and a closed state (e.g., in which fluid may not pass through flow regulator 141). In non-limiting embodiments, such flow regulator(s) 141 may allow for withdrawal of fluid from fluid line(s) 115 (e.g., to decrease volume, increase viscosity, and/or decrease viscosity) and/or addition of fluid to fluid line(s) 115 (e.g., to increase volume, increase viscosity, and/or decrease viscosity). In non-limiting embodiments, an amount of fluid in fluid line(s) 115 may be adjusted to allow for concentration or separation of, for example, cells in fluid line(s) 115.

In non-limiting embodiments, one or more sensors 140 may be arranged co-extensively with such flow regulator(s) 141, such that one or more parameters of the therapeutic composition may be analyzed prior to continued delivery through fluid line(s) 115. For example, in non-limiting embodiments, one or more sensors 140 may be arranged at or near a flow regulator 141, and may be configured to, with processor 124, detect cell rupture (for example, based on forces to which the therapeutic composition is exposed during delivery through fluid line(s) 115, including any needles, for example as described in Wahlberg et al., “Ex vivo biomechanical characterization of syringe-needle ejections for intracerebral cell delivery”, Scientific Reports 2018, 8: 9194) in the therapeutic composition. If the number of cells that have been ruptured reaches a certain threshold, one or more of the flow regulator(s) 141 may be opened to allow flow of the therapeutic composition containing the cells to be diverted to a separate fluid line 116 for further analyses and/or disposal. While FIG. 1 shows a certain orientation and arrangement of sensor(s) 140 and flow regulator(s) 141, those of skill in the art will appreciate that variations may be made.

In non-limiting embodiments, fluid line(s) 115 may include one or more filtering and/or microfluidics mechanisms for separating debris from the therapeutic composition and/or for concentrating the therapeutic composition. For example, fluid line(s) 115 may include one or more filters configured to restrict the flow of debris from ruptured cells from being introduced into the patient. Suitable, non-limiting embodiments of concentrating and/or filtering mechanisms include microfluidics (such as described in Martel et al., “Continuous Flow Microfluidic Bioparticle Concentrator”, Scientific Reports 2015, 5: 11300), fluorescence-activated cell sorting (FACS), flow-through cell concentrators, undulations in fluid line(s) 115, use of siphon lines (e.g., to separate fluid from cell-containing fluid), magnetic bead sorting, centrifugation, and/or microbubble sorting (e.g., available from Akadeum Life Sciences, Inc., Ann Arbor, MI). In non-limiting embodiments, fluid line(s) 115 may be a dual-lumen catheter, which may be used to concentrate the therapeutic composition during delivery and/or to deliver the therapeutic composition and/or a second therapeutic composition, as described herein. In non-limiting embodiments, the dual-lumen nature of fluid line(s) 115 extends to a delivery needle in fluid communication with fluid line(s) 115.

In non-limiting embodiments, where data received from sensors 140 is indicative of an adverse event, delivery device 120, through processor 124, may cause powered drive 122, or a second drive, to deliver a second therapeutic composition, for example, one to treat the adverse event. In non-limiting embodiments, data received from sensor(s) 140 may indicate that an immune response, such as a cytokine storm, is likely to occur or is occurring in the patient P. Non-limiting examples of a suitable sensor 140 for determining that an immune response, such as a cytokine storm, is occurring in the patient include those described in Xu et al., “Real-time Monitoring and Early Warning of a Cytokine Storm In Vivo Using a Wearable Noninvasive Skin Microneedle Patch”, Adv. Healthcare Materials 2023, 12(18): 2203133).

In non-limiting embodiments, if data received from sensor(s) 140 indicates that a cytokine storm is likely to occur or is occurring, processor 124 may initiate a flush with or delivery of, for example, a second therapeutic composition such as a crystalloid solution, a colloid solution, normal saline, immunosuppressants, beta agonists, epinephrine (or other adrenergic agonists), cytokines and/or cytokine inhibitors (e.g., IL-7 inhibitors), D5W (dextrose in water), Lactated Ringers solution, and/or a corticosteroid. In non-limiting embodiments, powered drive 122, or a second drive, may include fluid lines (or one or more additional lumens within fluid line(s) 115) that are co-extensive with fluid lines 115, and the flow regulator(s) 141 may be configured such that multiple configurations are possible, e.g., a closed position for fluid line(s) 115; an open position for fluid line(s) 115; a closed position for additional fluid lines (and/or lumens) connected to a flush/delivery container; and/or an open position for additional fluid line(s) (and/or lumens) connected to a flush/delivery container. Delivery of a second therapeutic composition may occur as an alternative to continued delivery of the therapeutic composition from container 110, and/or may occur in parallel with continued delivery of the therapeutic composition.

In non-limiting embodiments, processor 124, in response to determining that an immune response, such as a cytokine storm, is likely to occur or is occurring in the patient, and/or in response to a signal received indicating that a patient call button has been actuated, pauses delivery of the therapeutic composition. In non-limiting embodiments, processor 124 automatically pauses delivery of the therapeutic composition and/or causes delivery of a second therapeutic composition as described herein, through system 100 and/or through a different system with which system 100 may be in communication. In non-limiting embodiments, processor 124, in response to determining that a cytokine storm is likely to occur or is occurring in the patient, and/or in response to a signal received indicating that a patient call button has been actuated, causes a display and/or speaker to provide an indication that delivery of the therapeutic composition should be paused and/or stopped, and/or that delivery of a second therapeutic composition as described herein should be initiated. Processor 124 may also provide, for example through a display and/or speaker, one or more alternative mitigation strategies.

In non-limiting embodiments, system 100 may be in communication with a database storing one or more records of the patient, for example, one or more records of a prior treatment with a therapeutic composition, including, for example, an identification of the therapeutic composition, one or more side-effects, one or more thresholds of delivery of the therapeutic composition that results in the side effects (e.g., pressure, the flow rate, the number of ruptured cells, and/or the concentration of debris) and/or an identification of one or more second therapeutic compositions that are effective to mitigate the one or more side effects in the patient. In non-limiting embodiments, system 100 is configured to deliver one or more second therapeutics as described herein prior to delivery of the therapeutic composition. In non-limiting embodiments, system 100 is configured to provide an alert that one or more second therapeutics should be delivered.

Although the above devices, systems, and methods have been described in detail for the purpose of illustration based on what is currently considered to be the most practical and preferred embodiments or aspects, it is to be understood that such detail is solely for that purpose and that the present disclosure is not limited to the described embodiments or aspects but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiments or aspects.

1. A system for delivering a cell therapy to a patient, comprising:
a container configured to hold a therapeutic composition, the therapeutic composition comprising one or more cells in a suspension;
at least one powered drive in operative connection with the container, the at least one powered drive being configured to pressurize the therapeutic composition within the container and at least one fluid line in communication therewith;
a control system having a plurality of sensors and at least one processor programmed or configured to receive data from the plurality of sensors and, based at least in part on the data, to control the at least one powered drive so as to pressurize the therapeutic composition within the container and the at least one fluid line and thereby to inject the therapeutic composition into the patient at a first flow rate;
the plurality of sensors including at least two of:
a pressure sensor for measuring and providing data indicative of pressure within the container and the at least one fluid line,
a flow sensor for measuring and providing data indicative of an actual flow rate of the suspension within the at least one fluid line,
a counting sensor for providing data indicative of a count within the suspension flowing through the at least one fluid line of at least one of (i) a number of the one or more cells that remain whole and (ii) a number of the one or more cells that have ruptured,
a site sensor for placement about a site of injection of the suspension into the patient and for providing data indicative of one of an occurrence and a non-occurrence of an extravasation thereat,
a temperature sensor for measuring and providing data indicative of a temperature of the suspension within the at least one fluid line,
a chemical sensor for detecting and providing data indicative of an occurrence of an immune response in the patient, and
at least one physiological sensor for measuring and providing data indicative of at least one parameter of the patient;
wherein the at least one processor is further programmed or configured to control the at least one powered drive to pressurize the therapeutic composition within the container and thereby to inject the therapeutic composition into the patient at a second flow rate upon determining that the data received from the plurality of sensors indicate at least one of:
the pressure within at least one of the container and the at least one fluid line is greater than a predetermined pressure threshold;
the flow rate of the suspension within the at least one fluid line is greater than a predetermined flow rate threshold;
the number of the one or more cells within the suspension that have ruptured is greater than a predetermined rupture threshold;
the occurrence of an extravasation threat about the site of the injection;
the temperature of the suspension within the at least one fluid line is outside a predetermined temperature range;
the occurrence of an immune response in the patient as evidenced by the data received from at least one of the chemical sensor and the at least one physiological sensor; and
an adverse reaction experienced by the patient as evidenced by the data received from the at least one physiological sensor.

2. The system of claim 1, wherein the container is one of a syringe, a bag, a bottle and a vial.

3. The system of claim 1, wherein the one or more cells include at least one of: (i) autologous cells, (ii) allogenic cells, (iii) a combination of the autologous cells and the allogenic cells, (iv) a mixture in which at least one of the autologous cells and the allogenic cells have been modified, (v) genetically-engineered cells and (vi) genetically-engineered T-cells that express chimeric antigen receptors.

4. The system of claim 1, wherein one of the plurality of sensors is an ultrasound sensor and the suspension comprises an ultrasound contrast agent.

5. The system of claim 1, wherein the at least one powered drive is a pump system comprising an injector system.

6. The system of claim 1, wherein the second flow rate is one of a cessation of flow and a reduction in flow.

7. The system of claim 1, wherein the at least one physiological sensor comprises at least one of a blood pressure sensor, a heart rate sensor, a respiration sensor, a temperature sensor, and a chemical sensor.

8. The system of claim 1, wherein, upon determining that the patient is experiencing an adverse reaction, the at least one processor is programmed or configured to cause the at least one powered drive to deliver a second therapeutic composition to the patient.

9. The system of claim 8, wherein the second therapeutic composition comprises at least one of a crystalloid solution, a colloid solution, and a corticosteroid, wherein the crystalloid solution includes at least one of normal saline, D5W, and lactated Ringers.

10. The system of claim 1, wherein the at least one processor is further programmed or configured to trigger at least one of an alarm and an alert upon determining that the data received from the plurality of sensors indicate any of:
the pressure within at least one of the container and the at least one fluid line is greater than the predetermined pressure threshold;
the flow rate of the suspension within the at least one fluid line is greater than the predetermined flow rate threshold;
the number of the one or more cells within the suspension that have ruptured is greater than the predetermined rupture threshold;
the occurrence of an extravasation about the site of the injection;
the temperature of the suspension within the at least one fluid line is outside a predetermined temperature range;
the occurrence of an immune response in the patient as evidenced by the data received from at least one of the chemical sensor and the at least one physiological sensor; and
an adverse reaction experienced by the patient as evidenced by the data received from the at least one physiological sensor.

11. The system of claim 1, further comprising at least one flow regulator positioned within the at least one fluid line, each of the at least one flow regulators being configured to be switchable between an open state and a closed state such that upon:
determining that a volume of the therapeutic composition needs to be increased, the at least one processor is programmed or configured to switch the at least one flow regulator to the open state and thereby permit at least one of a diluent and a buffer to be added to the suspension via the at least one fluid line;
determining that the volume of the therapeutic composition needs to be reduced, the at least one processor is programmed or configured to switch the at least one flow regulator to the open state and thereby permit a portion of fluid within the suspension to be removed therefrom via the at least one fluid line; and
detecting that the number of the one or more cells that have ruptured has reached the predetermined rupture threshold, the at least one processor is programmed or configured to switch the at least one flow regulator to the open state and thereby divert the suspension to a separate fluid line of the at least one fluid line for at least one of analysis and disposal.

12. A system for delivering a cell therapy to a patient, comprising:
a container configured to hold a therapeutic composition, the therapeutic composition comprising one or more cells in a suspension;
at least one powered drive in operative connection with the container, the at least one powered drive being configured to pressurize the therapeutic composition within the container and at least one fluid line in communication therewith;
a plurality of sensors including at least two of (i) a pressure sensor for measuring and providing data indicative of pressure within the container and the at least one fluid line, (ii) a flow sensor for measuring and providing data indicative of an actual flow rate of the suspension within the at least one fluid line, (iii) a counting sensor for providing data indicative of a count within the suspension flowing through the at least one fluid line of at least one of (i) a number of the one or more cells that remain whole and (ii) a number of the one or more cells that have ruptured, (iv) a site sensor for placement about a site of injection of the suspension into the patient and for providing data indicative of one of an occurrence and a non-occurrence of an extravasation thereat, (v) a temperature sensor for measuring and providing data indicative of a temperature of the suspension within the at least one fluid line, (vi) a chemical sensor for detecting and providing data indicative of an occurrence of an immune response in the patient, ; and (vii) at least one physiological sensor for measuring and providing data indicative of at least one parameter of the patient; and
at least one processor programmed or configured to receive the data from the plurality of sensors and, based at least in part on the data, to control the at least one powered drive so as to pressurize the therapeutic composition within the container and the at least one fluid line and thereby to inject the therapeutic composition into the patient at a desired flow rate whose magnitude depends on the data received from the plurality of sensors.

13. The system of claim 12, wherein the desired flow rate is initially a first flow rate but changes to a second flow rate when the data received by the at least one processor from the plurality of sensors indicate at least one of:
the pressure within at least one of the container and the at least one fluid line is greater than a predetermined pressure threshold;
the flow rate of the suspension within the at least one fluid line is greater than a predetermined flow rate threshold;
the number of the one or more cells within the suspension that have ruptured is greater than a predetermined rupture threshold;
the occurrence of an extravasation about the site of the injection;
the temperature of the suspension within the at least one fluid line is outside a predetermined temperature range;
the occurrence of an immune response in the patient as evidenced by the data received from at least one of the chemical sensor and the at least one physiological sensor; and
an adverse reaction experienced by the patient as evidenced by the data received from the at least one physiological sensor.

14. The system of claim 12, wherein the one or more cells include at least one of (i) autologous cells, (ii) allogenic cells, (iii) a combination of the autologous cells and the allogenic cells, (iv) a mixture in which at least one of the autologous cells and the allogenic cells have been modified, (v) genetically-engineered cells and (vi) genetically-engineered T-cells that express chimeric antigen receptors.

15. The system of claim 12, wherein one of the plurality of sensors is an ultrasound sensor and the suspension comprises an ultrasound contrast agent.

16. The system of claim 12, wherein the at least one powered drive is a pump system comprising an injector system.

17. The system of claim 12, wherein the second flow rate is one of a cessation of flow and a reduction in flow.

18. The system of claim 12, wherein the at least one physiological sensor comprises at least one of a blood pressure sensor, a heart rate sensor, a respiration sensor, a temperature sensor and a chemical sensor.

19. The system of claim 12, wherein, upon determining that the patient is experiencing an adverse reaction, the at least one processor is programmed or configured to cause the at least one powered drive to deliver a second therapeutic composition to the patient.

20. The system of claim 19, wherein the second therapeutic composition comprises at least one of a crystalloid solution, a colloid solution, and a corticosteroid, wherein the crystalloid solution includes at least one of normal saline, D5W, and lactated Ringers.

21. The system of claim 12, wherein the at least one processor is further programmed or configured to trigger at least one of an alarm and an alert upon determining that the data received from the plurality of sensors indicate any of:
the pressure within at least one of the container and the at least one fluid line is greater than a predetermined pressure threshold;
the flow rate of the suspension within the at least one fluid line is greater than a predetermined flow rate threshold;
the number of the one or more cells within the suspension that have ruptured is greater than a predetermined rupture threshold;
the occurrence of an extravasation about the site of the injection;
the temperature of the suspension within the at least one fluid line is outside a predetermined temperature range;
the occurrence of an immune response in the patient as evidenced by the data received from at least one of the chemical sensor and the at least one physiological sensor; and
an adverse reaction experienced by the patient as evidenced by the data received from the at least one physiological sensor.

22. The system of claim 12, further comprising at least one flow regulator positioned within the at least one fluid line, each of the at least one flow regulator being configured to be switchable between an open state and a closed state such that upon:
determining that a volume of the therapeutic composition needs to be increased, the at least one processor is programmed or configured to switch the at least one flow regulator to the open state and thereby permit at least one of a diluent and a buffer to be added to the suspension via the at least one fluid line;
determining that the volume of the therapeutic composition needs to be reduced, the at least one processor is programmed or configured to switch the at least one flow regulator to the open state and thereby permit a portion of fluid within the suspension to be removed therefrom via the at least one fluid line; and
detecting that the number of the one or more cells that have ruptured has reached a predetermined rupture threshold, the at least one processor is programmed or configured to switch the at least one flow regulator to the open state and thereby divert the suspension to a separate fluid line of the at least one fluid line for at least one of analysis and disposal.

23-31. (canceled)
